(19) United States
(12) Patent Application Publication
(10) Pub. No.: US 2025/0017481 A1
(43) Pub. Date: Jan. 16, 2025

(54) APPARATUS AND METHODOLOGY FOR VESSEL-CONTACTED ACCELEROMETER-BASED HEMODYNAMIC MONITORING SYSTEM

(71) Applicant: THE TRUSTEES OF THE STEVENS INSTITUTE OF TECHNOLOGY, Hoboken, NJ (US)

(72) Inventors: Arash Shokouhmand, Weehawken, NJ (US); Negar Ebadi, Menlo Park, CA (US)

(73) Assignee: THE TRUSTEES OF THE STEVENS INSTITUTE OF TECHNOLOGY, Hoboken, NJ (US)

(21) Appl. No.: 18/416,443

(22) Filed: Jan. 18, 2024

Related U.S. Application Data
(60) Provisional application No. 63/440,068, filed on Jan. 19, 2023.

(51) Int. Cl. A61B 5/0295; A61B 5/00; A61B 5/02; A61B 5/0205; A61B 5/021; A61B 5/107; A61B 5/11; A61B 5/28

(52) U.S. Cl. CPC A61B 5/0295; A61B 5/02007; A61B 5/02108; A61B 5/02133; A61B 5/1075; A61B 5/1121; A61B 5/28; A61B 5/681; A61B 5/7264; A61B 5/746; A61B 5/0205; A61B 2560/0462; A61B 2562/0204; A61B 2562/0219

(57) ABSTRACT

A sensor for blood-based measurement parameters that can be integrated on any flat surface to allow for ubiquitous monitoring of hemodynamics is disclosed. Such a sensor can measure heart rate, heart rate variability, blood pressure, etc. The sensor is adapted to be placed in direct contact with blood vessels. This can be done with an accelerometer or another type of vibrational or acoustic sensor in order to measure arterial vibrations.

### CROSS-REFERENCE TO RELATED APPLICATION

This application claims priority to U.S. Provisional Patent Application Ser. No. 63/440,068 filed Jan. 19, 2023, the entire disclosure of which is incorporated herein by reference.

### STATEMENT REGARDING FEDERALLY SPONSORED RESEARCH

This invention was made with government support under Grant No. 1855394 awarded by the National Science Foundation. The U.S. government has certain rights in the invention.

### FIELD OF THE INVENTION

The present invention relates to monitoring hemodynamic features through direct contact with blood vessels.

### BACKGROUND OF THE INVENTION

A wide range of cardiovascular abnormalities such as hypertension, hypotension, and arrhythmia require ubiquitous monitoring to prevent adverse outcomes. Although smart watches (and other wearable sensors) could provide certain hemodynamic features, they are noticeable in an unwelcome manner.

Present technologies for hemodynamic monitoring include electrocardiogram (ECG) and photoplethysmography (PPG) sensors which estimate heart rate based on electrical and optical modalities respectively. Present technologies for blood pressure estimation are either cuff-based, calibrated using the cuff, or invasive. Pulse transit time (PTT) has been recently investigated for the estimation of blood pressure. This method, however, lacks accuracy due to variations in the morphology of the cardiac cycle among people.

Present technologies which are in the form of wearable sensors tend to be obtrusive. Other technologies (e.g., point-of-care devices) can be intrusive and bulky due to the presence of wires and cables.

Cuff-based systems use a cuff to ensure equal transmural pressure between the outside and inside of the vessel. Home-based blood pressure monitors typically operate using a cuff equipped with a pressure sensor to provide a snapshot measurement of blood pressure and heart rate. The use of a cuff generally results in an obtrusive system, and the inconvenience caused by the cuff makes patients reluctant to measure their blood pressure as frequently as prescribed by physicians. As a result, their diseases may progress into more severe and uncontrollable cases, eventually leading to adverse outcomes

For the pulse wave velocity (PWV)-based method, a combination of two or more of the electrocardiogram (ECG), photo-plethysmography (PPG), impedance cardiogram (ICG), seismocardiogram (SCG), and gyrocardiogram (GCG) sensors are used to relate pulse transit time (PTT) or pulse arrival time (PAT) to the blood pressure. This setting requires the use of multiple sensors which makes such systems inconvenient.

### SUMMARY OF THE INVENTION

Unlike the prior art discussed above, the present technology is able to be unobtrusively integrated on any flat surface, allowing for ubiquitous monitoring of hemodynamics. The present invention is a novel hardware setup and methodology for monitoring hemodynamic features through direct contact of an accelerometer with blood vessels. Direct contact of an accelerometer with blood vessels is a novelty which allows for monitoring hemodynamics in an unobtrusive manner.

Unlike techniques based on a pressure sensor embedded in an inflatable cuff, or the estimation of pulse wave velocity (PWV), the present invention does not use any cuff or multiple-sensor configuration. It only uses an accelerometer in direct contact with the blood vessel to pick up arterial acceleration caused by the blood. This acceleration is then translated to a pressure wave using Newton's law.

Unlike the methods mentioned above, the disclosed invention estimates hemodynamics based on arterial vibrations. The present invention does not require a cuff since it measures the systolic and diastolic blood pressure directly from, for instance, an artery, and it provides highly accurate measurement of blood pressure as a result of direct contact between the sensor and the artery. Direct contact allows for cuff-less blood pressure monitoring and permits highly accurate estimation of blood pressure. Moreover, the disclosed technology is a small monitoring system that can be seamlessly integrated with life routines.

It is an object of the present invention to offer continuous monitoring of heart rate, heart rate variability, blood pressure, etc. For instance, the sensing platform can be embedded in smart watches to estimate hemodynamic features on a second-by-second basis. Another application is hemodynamic monitoring in a clinic: the sensing platform can be integrated with any flat surfaces in the clinic for monitoring blood pressure, heart rate, etc. Another objective of the present invention is to provide for continuous monitoring of a driver's health status, including hemodynamic features and stress levels, during driving.

In some embodiments, the present technology can be a hemodynamic monitoring device for home-based and/or clinical settings. The technology could also be sold as an add-on to any daily use apparatus with a flat surface on which people place and hold their hands. For instance, keyboard manufacturers, furniture manufacturers, wearable device manufacturers, and medical device companies may integrate the present technology with their products as an add-on to provide clinical grade, comprehensive assessment of health monitoring.

In an embodiment, the healthcare system can benefit from the inventive device by allowing for the monitoring of health status of individuals. The military could also use the present technology to monitor soldiers' health status in the field. Similarly, the National Aeronautics and Space Administration (NASA) can use the present technology to monitor the health status of astronauts in space.

Further applications of the present invention can also extend to detection of arterial (venous) obstructions, estimation of vessel diameter, a warning system for imminent stroke and a warning system for cardiac arrhythmia.

### BRIEF DESCRIPTION OF THE DRAWINGS

For a more complete understanding of the present invention, reference is made to the following detailed description of various embodiments considered in conjunction with the accompanying drawings, in which:

FIG. 1 is a schematic illustration showing the setup of a sensing device in accordance with an embodiment of the present invention;

FIG. 2 is a block diagram illustrating the interaction of hardware in a device made in accordance with an embodiment of the present invention; and

FIG. 3 is a schematic diagram illustrating the estimation of blood pressure and other hemodynamic parameters in accordance with an embodiment of the present invention.

### DETAILED DESCRIPTION OF THE EXEMPLARY EMBODIMENT

The following disclosure is presented to provide an illustration of the general principles of the present invention and is not meant to limit, in any way, the inventive concepts contained herein. Moreover, the particular features described in this section can be used in combination with the other described features in each of the multitude of possible permutations and combinations contained herein.

All terms defined herein should be afforded their broadest possible interpretation, including any implied meanings as dictated by a reading of the specification as well as any words that a person having skill in the art and/or a dictionary, treatise, or similar authority would assign thereto. Further, it should be noted that, as recited herein, the singular forms “a”, “an”, “the”, and “one” include the plural referents unless otherwise stated. Additionally, the terms “comprises” and “comprising” when used herein specify that certain features are present in that embodiment, however, this phrase should not be interpreted to preclude the presence or addition of additional steps, operations, features, components, and/or groups thereof.

All examples and conditional language recited herein are intended for pedagogical purposes to aid the reader in understanding the principles of the invention and the concepts contributed thereby to furthering the relevant art, and are to be construed as being without limitation to such specifically recited examples and conditions. Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Arterial obstructions cause blood flow to be reduced or stopped. Reduction in blood flow is expected to generate weaker vibrations on the arterial wall compared to the case of normal blood flow. The patterns corresponding to weak vibrations can be compared to those from normal flow to detect obstructions in the artery. Vascular walls act as dampers for the vibrations produced by the blood flow. The greater the diameter of the vessel, the weaker the vibration on the vessel. Therefore, vibrational components on the vascular wall in people with thicker vessels are weaker in comparison to people with thinner vessels. Depending on the energy of the vibrational signal captured by an accelerometer, the diameter can be estimated. Additionally, elevated blood pressure precedes blood stroke. These readings can therefore be used to inform people of an imminent blood stroke. The present invention can be incorporated (e.g., retrofitted) into any flat surface, including table tops, arms of chairs, keyboards, coffee mugs, etc. It can also be incorporated in smart watches and in clothing such as gloves to continuously estimate blood pressure.

Cardiac arrhythmia requires continuous monitoring of the heart cycle. The present invention provides useful information about the heart rate and beat-to-beat cardiac cycle through sensing vibrational components on the vessel wall. Hence, arrhythmia can be detected using irregular heartbeat patterns.

The present invention is a composition of measurement hardware and methodology. The measurement hardware includes an accelerometer sensor and an analog-to-digital (A2D) circuit, consolidated into a small module directly placed on a vessel (e.g., fingertip, radial artery, carotid artery, etc.) to capture arterial/venous vibrations. The direct contact between the sensor and vessel allows for capturing vibrational components produced by the interaction between blood flow and the vascular wall. These vibrations represent successive cardiac cycles, from which hemodynamic features can be estimated. The sensor module transfers the collected data to a computer via a data acquisition module. Hardware design and sensor placement on the vessel, respectively, constitute a device and a process.

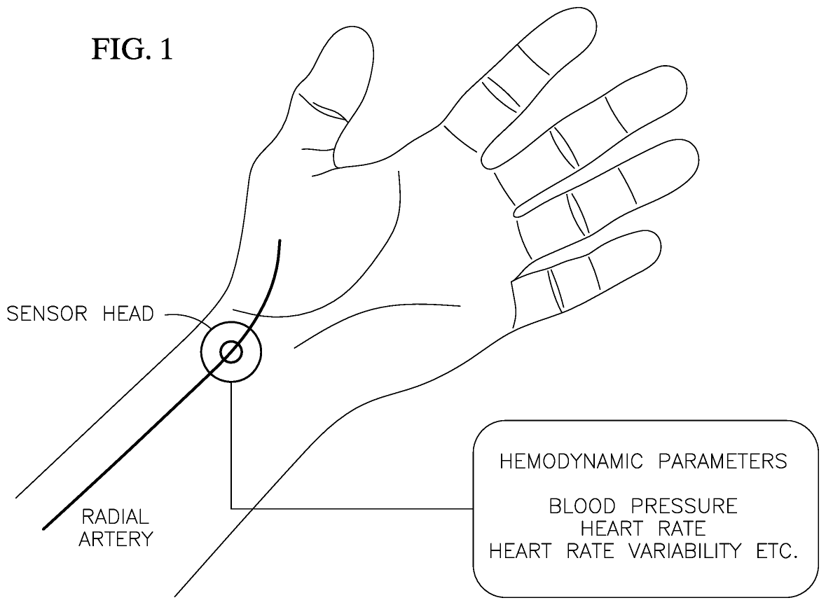

As shown in FIG. 1, the sensor head includes an accelerometer (or any other vibrational or acoustic sensor) which is placed directly on a vessel (an example of a radial artery is shown in FIG. 1). The accelerometer captures the vibrational components corresponding to the interaction of blood flow with the vascular wall to monitor hemodynamic features including blood pressure, heart rate (HR), heart rate variability (HRV), cardiac intervals, etc. In this sensing technology, other types of sensors such as electrocardiogram (ECG), photoplethysmogram (PPG), gyrocardiogram (GCG), etc. can be used to augment data, and extract more hemodynamic features with higher accuracies.

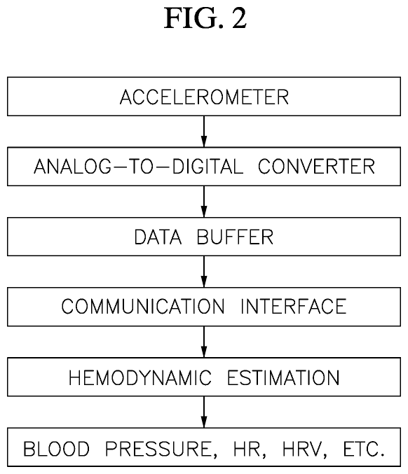

As depicted in FIG. 2, the sensor head includes an accelerometer coupled with an analog-to-digital (A2D) converter to digitize the vibrational data. The data is then buffered in a memory which allows for transferring data through a communication interface (which can be through a cable or Bluetooth connection) to a data processing unit. The data processing unit (which can be a microcontroller, microprocessor, or any other processing module) processes the data to estimate hemodynamic features using signal processing, machine learning, and/or statistical analysis techniques.

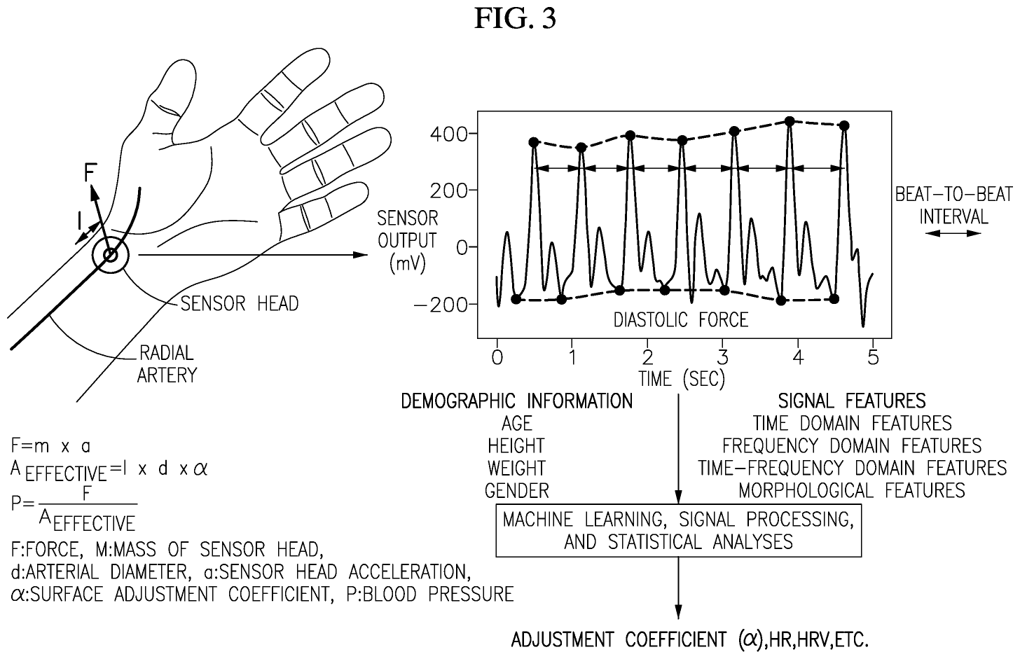

A signal sample is shown in FIG. 3. The output voltage of the accelerometer is proportional to the acceleration with which the vessel moves following the interaction between blood flow and the vessel. Using Newton's second law, the force (F) imposed on the sensor head can be calculated by multiplying the acceleration (a) by the mass of the sensor head (m):

F=ma

The effective area of contact between the sensor and the vessel (A_{effective}) is dependent on the length of the sensor head (l) and the diameter of the vessel (d):

$${A}_{effectve} = l \times d \times a$$

As the surfaces of the skin and vessel are not flat, an adjustment coefficient (α) is considered to accurately quantify the effective area. Once the contact area is estimated, blood pressure (P) can be determined using Pascal's law (by dividing the force by the effective surface):

$$P = \frac{F}{{A}_{effective}}$$

Time-domain, frequency-domain, time-frequency-domain, and morphological features are used as input to a model to determine the adjustment coefficient. The model could be developed using machine learning models, signal processing techniques, statistical analyses, or a combination of all of these.

The morphology of the signal can also be used to extract other hemodynamic parameters such as heart rate (HR), heart rate variability (HRV), blood volume, cardiac time intervals, etc. Demographic information about the subject can augment the feature space for the estimation of hemodynamic parameters.

The disclosed invention may be capable of accurately monitoring hemodynamic features with an accuracy of 91% compared to current “gold standards” (i.e., electrocardiogram (ECG) and TL-300 for heart rate and blood pressure monitoring, respectively).

It will be understood that the embodiments described herein are merely exemplary and that a person skilled in the art may make many variations and modifications without departing from the spirit and scope of the invention. All such variations and modifications are intended to be included within the scope of the invention.

1. A device for measuring blood-related parameters, said device comprising:
a sensor module adapted to measure vibrational or acoustic signals from a patient's blood flow;
a data-acquisition unit, communicatively coupled to said sensor module, and adapted to digitize said vibrational or acoustic signals;
a memory unit, communicatively coupled to said data-acquisition unit, and adapted to buffer and save said digitized vibrational or acoustic signals; and
a data-processing unit, communicatively coupled to said memory unit, and adapted to convert said digitized vibrational or acoustic signals into one or more measured parameters.

2. The device of claim 1, wherein said sensor module is adapted to be placed in direct contact with the patient's blood vessels.

3. The device of claim 1, wherein said sensor module consists of an accelerometer.

4. The device of claim 1, wherein said data-processing unit is adapted to calculate blood pressure, including systolic and diastolic blood pressure values; cardiac intervals; heart rate variance; or heart rate.

5. The device of claim 1, wherein said device is integrated on a smart watch.

6. The device of claim 5, wherein said sensor module is adapted to be placed in direct contact with the patient's blood vessels.

7. The device of claim 1, wherein said data-processing unit is adapted to detect arterial or venous obstructions by comparing said vibrational or acoustic signals with readings from expected blood flow.

8. The device of claim 1, wherein said data-processing unit is adapted to estimate vessel diameter.

9. The device of claim 1, further comprising a warning system for stroke or arrhythmia.

10. The device of claim 1, wherein said sensor module further comprises an electrocardiogram sensor; a gyrocardiogram sensor; or a photoplethysmogram sensor.

11. The device of claim 1, wherein said data-processing unit is adapted to obtain acceleration values from the digitized vibrational or acoustic signals.

12. The device of claim 11, wherein said data-processing unit is further adapted to convert said acceleration values into a pressure wave.

13. A method for measuring blood flow parameters, comprising the steps of:
placing a vibrational or acoustic sensor in direct contact with a patient's blood vessels;
obtaining digital acceleration measurements from said vibrational or acoustic sensor; and
converting said digital acceleration measurements into a pressure wave.

14. The method of claim 13, wherein said sensor comprises an accelerometer.

15. The method of claim 13, wherein said sensor comprises a sensor selected from the group consisting of: an electrocardiogram sensor; a gyrocardiogram sensor; and a photoplethysmogram sensor.

16. The method of claim 13, further comprising the steps of comparing said pressure wave with readings of normal blood flow and determining if there is an obstruction in the patient's blood vessels.

17. The method of claim 13, further comprising the step of obtaining a heart rate value; a blood pressure value; a cardiac interval value; or a heart rate variability value from said pressure wave.

18. The method of claim 13, further comprising the step of estimating the patient's blood vessel's diameter from said pressure wave.

19. The method of claim 13, further comprising the steps of detecting elevated blood pressure from said pressure wave; and alerting the patient regarding a potential stroke.

20. The method of claim 13, further comprising the steps of detecting irregular heartbeat patterns from said pressure wave; and alerting the patient regarding a potential arrhythmia.
